(19) United States
(12) Patent Application Publication
(10) Pub. No.: US 2009/0227995 A1
(43) Pub. Date: Sep. 10, 2009

(54) Precision Tube Assembly

(76) Inventors: Jayant D. Bhawalkar, Chelmsford, MA (US); Christopher J. Jones, Leicester, MA (US); Herbert R. Otterson, Needham, MA (US)

Correspondence Address: PROSKAUER ROSE LLP, ONE INTERNATIONAL PLACE, BOSTON, MA, 02110, US

(21) Appl. No.: 12/400,740

(22) Filed: Mar. 9, 2009

Related U.S. Application Data
(63) Continuation-in-part of application No. 11/865,365, filed on Oct. 1, 2007.
(60) Provisional application No. 60/848,083, filed on Sep. 29, 2006.

(51) Int. Cl. A61B 18/20

(52) U.S. Cl. 606/9; 606/10

(57) ABSTRACT

Described are laser systems for treating skin. The system includes a first solid-state laser for producing a first output beam, a second solid state laser for producing a second output beam, and a delivery device for directing the second output beam to a target region of skin. The second solid state is adapted to receive a first part of the first output beam and generate excitation in a rare-earth doped gain medium to produce the second output beam. The second output beam is for treating the skin.

### CROSS-REFERENCE TO RELATED APPLICATION

This application is a continuation-in-part of U.S. patent application Ser. No. 11/865,365 filed Oct. 1, 2007, which claims the benefit of and priority to U.S. Provisional Application Ser. No. 60/848,083 filed Sep. 29, 2006, which is owned by the assignee of the instant application and the disclosure of which is incorporated herein by reference in its entirety.

### FIELD OF THE INVENTION

The invention relates generally to a device for skin treatment using radiation. More particularly, the invention relates to a device for precision alignment of the output coupler and the high reflector of a laser resonator.

### BACKGROUND OF THE INVENTION

Lasers are widely used in dermatological applications such as hair removal, removal of pigmented lesions, tattoos, vascular lesions, wrinkles, acne, and skin tightening. Dermatological laser treatments are typically based on selective targeting of a chromophore in the skin by an appropriate choice of wavelength and pulse duration of the laser light. Although lasers can provide better results than most other light sources, most medical laser devices use only a single wavelength of light. This limits the range of applications for which a particular medical laser can be used. Therefore, several different lasers can be needed to treat more than one skin condition.

In addition, solid-state lasers are typically pumped by flashlamps to get the large pulse energies required for creating the desired thermal profile in the skin. While such lasers can provide large output energies, the beam quality is generally poor and frequency conversion can be difficult.

Solid state lasers can be used to excite a second laser. Multi-laser systems based on laser pumping of one or more other lasers can provide two or more wavelengths with little additional cost, complexity or size. Also, a laser pumped laser generally can have better beam quality than a flashlamp pumped laser. Better beam quality can permit the generation of additional wavelengths by various methods of non-linear frequency conversion. Furthermore, laser pumping is particularly advantageous for lasers that are difficult to pump with other conventional sources like laser diodes and flashlamps.

Precision alignment in laser pumped laser systems can be difficult and require a high level of skill. In addition, even after acceptable alignment is achieved, the alignment is susceptible to drift, even without mechanical shock or vibration.

### SUMMARY OF THE INVENTION

The invention features, in one embodiment, a precision tube design that achieves alignment of the output coupler and the high reflector of a laser resonator. The precision tubes can achieve the required parallelism of the mirrors not by mechanical adjustment by the assembler, but rather by registering the mirrors against a tube that has parallel faces or faces situated at a predetermined angle. The parallelism requirement can be extremely high, e.g., five arc seconds, translating to an error of only 0.25 microns in length of the tube across a 10 mm diameter.

In one aspect, the invention features a laser handpiece including a tubular member having a first end surface and a second end surface, a first optical component, and a second optical component. At least one raised region is formed on each end surface of the tubular member. The first optical component is engageable with the at least one raised region associated with the first end surface of the tubular member, and the second optical component is engageable with the at least one raised region associated with the second end surface of the tubular member. The tubular member is adapted to align the first optical component and the second optical component parallel or substantially parallel. In certain embodiments, the raised region(s) is adapted to align the first optical component and the second optical component to within about 5 arc seconds.

In another aspect, the invention features an alignment tube for a laser handpiece including a tubular member and at least one raised region formed on an end circumference of the tubular member. The at least one raised region is adapted to align an optical component in the laser handpiece to better than about five arc seconds.

In other examples, any of the aspects above or any apparatus, system or device or any method, process or technique described herein can include one or more of the following features.

In some embodiments, the raised region(s) includes a plurality of raised regions spaced substantially evenly around each end surface of the tubular member. The tubular member can include a channel engageable with an alignment feature of the laser handpiece to register faces of the first optical component and the second optical component parallel or substantially parallel.

In certain embodiments, the raised region(s) on the first end surface and the second end surface can define planes that are parallel or substantially parallel. The plane can be lapped parallel to better than five arc seconds.

The laser handpiece can include a washer pressing against a spacer to provide a force against an optical component to contact between the optical component and the at least one raised region of the tubular member. A locking ring can engage the laser handpiece to apply force to the washer.

In some embodiments, the first optical component is a high reflector and the second optical component is an output coupler. A Brewster plate can be disposed within the tubular member.

A first solid state laser can be used to excite a second solid state laser for treatment of skin disorders and conditions. Excitation of one laser by another enables generation of a new wavelength, along with an increase in brightness which further allows non-linear frequency conversion. The increase in brightness can also allow the beam to be focused to a small spot of high intensity laser energy that can be used to cut tissue in surgical applications.

The laser handpiece can be used to treat mammalian tissue. A first output beam of laser radiation can be generated using a first solid-state laser, and a first part of the first output beam can be directed to a second solid-state laser. A second output beam of laser radiation can be generated using the second solid-state laser based on excitation of a rare-earth doped gain medium, and the second output beam can be directed to a target region of mammalian tissue to treat a first condition of the mammalian tissue.

A laser system can include a first solid-state laser for producing a first output beam and a second solid state laser for producing a second output beam. The second solid state laser is adapted to receive a first part of the first output beam and generate excitation in a rare-earth doped gain medium to produce the second output beam. A delivery device can direct the second output beam to a target region of skin, wherein the second output beam is for treating the skin.

In various embodiments, the mammalian tissue can be skin. In one embodiment, treating the first condition can include removing black tattoos. In various embodiments, the laser system can further include beam shaping optics. The beam shaping optics can be used to direct the first part of the first output beam to the second solid-state laser. In one embodiment, the laser system can further include an optical fiber. The optical fiber can be used to direct the first part of the first output beam to the second solid-state laser. In some embodiments, the laser system further includes a handpiece. The handpiece can be used to direct the first part of the second output beam to the target region. In one embodiment, the method can further include directing a second part of the first output beam to the target region to treat a second condition. The second condition can include removing violet tattoos, blue tattoos, green tattoos, black tattoos, or any combination thereof.

In various embodiments, the second solid state laser system can further include an output coupler mirror designed to transmit a second part of the first output beam. The output coupler mirror can transmit a second part of the first output beam. The output coupler mirror can form a dual wavelength output beam from a second part of the first output beam that passes through the second solid-state laser and laser radiation from the second solid-state laser. In one embodiment, the method can further include directing the dual wavelength output beam to the target region to treat the first condition and a second condition. In various embodiments, the method can include generating, based on laser radiation received from the second solid-state laser, the second output beam using a nonlinear frequency converter. The laser system can include the nonlinear frequency converter. Treating the third condition can include removing red tattoos, orange tattoos, yellow tattoos, or any combination thereof. In some embodiments, the laser system can further include a q-switching element in the first solid-state laser to generate high peak power pulses of the first output beam.

In various embodiments, the laser system can further include beam shaping optics adapted to receive laser radiation from the first solid-state laser for directing the first part of the first output beam to the second solid-state laser. The laser system can further include an optical fiber adapted to receive laser radiation from the first solid-state laser for directing the first part of the first output beam to the second solid-state laser. The delivery device can include a handpiece and the second solid state laser can be housed in the handpiece. The delivery device can include an output coupler mirror for forming a dual wavelength output beam from a second part of the first output beam that passes through the second solid-state laser and laser radiation from the second solid-state laser. In some embodiments, the laser system can further include a nonlinear frequency converter for generating, based on laser radiation received from the second solid-state laser, the second output beam. The laser system can further include a q-switching element in the first solid-state laser for generating high peak power pulses of the first output beam. The first solid-state laser can include a first host material. The first host material can include: sapphire, beryl, chrysoberyl, LiSAF, forsterite, or any combination thereof. The first host material can be doped with a transition metal, the transition metal comprising Cr or Ti. The second solid state laser can include a rare-earth doped gain medium. The rare earth doped gain medium can include: YAG, YAP, YVO4, YLF, YSGG, GSGG, FAP, GdVO₄, KGd(WO₄)₂, SFAP, glass, ceramic, or any combination thereof. The rare-earth doped gain medium can be doped with rare-earth ions. Rare-earth ions can include: Nd, Yb, Er, Ho, Th, Sm, Ce, or any combination thereof.

A laser system can be for tattoo removal. The laser system can include a Q-switched alexandrite laser for producing a first output beam, a Nd:YAG laser for producing a second output beam, and a delivery device for directing the second output beam to a target region of skin. The wavelength of the first output beam is about 755 nm. The wavelength of the second output beam is about 1064 nm. The Nd:YAG laser is adapted to receive the first output beam and generate excitation to produce the second output beam. The second output beam is for removing black tattoos.

A laser system for tattoo removal can include a Q-switched alexandrite laser for producing a first output beam, a Nd:YAG laser for producing a second output beam, a KTP crystal for generating a third output beam, and a delivery device for directing the third output beam to a target region of skin. The wavelength of the first output beam is about 755 nm. The wavelength of the second output beam is about 1064 nm. The Nd:YAG laser is adapted to receive the first output beam and generate excitation to produce the second output beam. The KTP crystal is adapted to receive the second output beam and generate a third output beam. The wavelength of the third output beam is about 532 nm. The third output beam is for removing red tattoos, orange tattoos, yellow tattoos, or any combination of tattoos.

In various embodiments, the first solid-state laser can include a transition-metal doped gain medium. In one embodiment, the first solid-state laser gain medium can be Alexandrite. In various embodiments, the laser system can further include a nonlinear frequency converter for generating a third output beam from at least a part of the second output beam. The nonlinear frequency converter can be a second harmonic generation converter, a third harmonic generation converter, a fourth harmonic generation converter, an optical parametric oscillator, or a Raman shifting converter. In some embodiments, the first solid-state laser can include a first host material. The second solid-state laser can include a second host material. The second host material can include a crystalline structure. The crystalline structure can include: YAG, YAP, YVO₄, YLF, YSGG, GSGG, FAP, GdVO₄, KGd(WO₄)₂, or SFAP. The second host material can include an amorphous structure. The amorphous structure can include: glass or ceramic YAG. The second host material can be doped with a rare-earth ion selected from: Nd, Yb, Er, Ho or Th. The doping of the second host material can include a rare-earth ion selected from: Nd, Yb, Er, Ho or Th. The doping of the second host crystal can include co-doping with one or more ions selected from: Cr, Nd, Yb, Er, Ho or Th. The rare-earth doped gain medium can be Nd:YAG.

The second output beam can include a single pulse or a train of pulses, each pulse of duration between approximately 1 ns and approximately 500 ms. Each pulse can have an energy between about 1 microJoule and about 100 Joules. The first beam output can include a wavelength between about 400 nm and about 1000 nm. The second beam output can include a wavelength between about 400 nm and about 3000 nm.

A laser system for treating skin can include a transition-metal laser producing a first output beam, and a rare-earth laser having a gain medium that receives at least a first part of the first output beam and generates excitation in the gain medium to produce a second output beam.

A laser system for treating skin can include a flashlamp, a first gain medium excited by the flashlamp for producing a first output beam, a second gain medium excited by a part of the first output beam for producing a second output beam, a first coupling element for coupling the part of the first output beam to the second gain medium, and a second coupling element for coupling a part of the second output beam out of a cavity containing the second gain medium.

A multi-wavelength laser system for treating skin can include a flashlamp pumped Alexandrite laser producing a first output beam having a first wavelength and a first beam path, and an Alexandrite-pumped neodymium laser. The Alexandrite-pumped neodymium laser is movable from a first position not in the first beam path to a second position in said first beam path. The Alexandrite-pumped neodymium laser is also capable of receiving at least some of the first output beam and producing a second output beam having a second wavelength and a second beam path coaxial with the first beam path. The neodymium laser includes a neodymium doped laser gain material.

In various embodiments, the Alexandrite laser can include a KD*P q-switch, producing high peak power pulses. The laser system can further include a KTP second harmonic generator movable from a first position not in the second beam path to a position in the second beam path producing a third output beam having a third output wavelength and a third beam path coaxial with the second beam path. The neodymium laser can further include a Cr⁴⁺:YAG passive q-switch where the second output beam comprises a train of high peak power pulses. The laser system can further include a KTP second-harmonic generator movable from a first position not in the second beam path to a position in the second beam path producing a third output beam comprising a train of high peak power pulses having a third output wavelength and a third beam path coaxial with the second beam path.

In various embodiments, the Alexandrite laser can include a KD*P q-switch, producing high peak power pulses. The handpiece can further include a KTP second-harmonic generator positioned in the second beam path, producing a third output beam having a third wavelength. The neodymium laser can further include a Cr⁴⁺:YAG passive q-switch producing a train of high peak power pulses, where the handpiece further comprises a KTP second-harmonic generator positioned in the second beam path, producing a third output beam having a third wavelength.

The details of one or more examples are set forth in the accompanying drawings and the description below. Further features, aspects, and advantages of the invention will become apparent from the description, the drawings, and the claims.

### BRIEF DESCRIPTION OF THE DRAWINGS

The advantages of the invention described above, together with further advantages, may be better understood by referring to the following description taken in conjunction with the accompanying drawings. The drawings are not necessarily to scale, emphasis instead generally being placed upon illustrating the principles of the invention.

FIG. 1 is a schematic drawing of a solid state laser pumped by a second solid state laser with q-switch and frequency converting elements.

FIG. 2 is a schematic drawing of a handpiece including a solid state laser pumped via an optical fiber by another solid state laser.

FIG. 3 is a graph of data from an experimental test of a rare-earth laser pumped by a transition metal laser.

FIG. 4 is a schematic drawing of a laser handpiece including a precision tube assembly.

FIG. 5 is a schematic drawing of a tubular member of a precision tube assembly.

FIG. 6 is another schematic drawing of a laser handpiece including a precision tube assembly.

### DESCRIPTION OF THE INVENTION

Lasers and other light sources are often used for the treatment of skin disorders and to produce cosmetic improvement in the appearance of the skin including the removal of hair from the skin. The heat produced by the light energy can modify structures within the skin and beneath the skin. Typical applications can include, for example, removal of hair, pigmented lesions, tattoos, vascular lesions, wrinkles, acne, skin tightening, and/or the like. Applications can more generally also include treatment of mammalian tissue.

Dermatological laser treatments can be based on selectively targeting of a chromophore in or near the target structure by an appropriate choice of wavelength and pulse duration of the light. Lasers are often the preferred light source because a laser beam has a narrower wavelength bandwidth than light from other sources. A source with a narrow wavelength bandwidth can maximize the spectral selectivity of the target chromophore. In addition, lasers can be made with much shorter pulse durations than other light sources thereby maximizing the temporal selectivity of the targeted structure. The superior temporal selectivity makes lasers especially preferred for removing small targets like small vessels and tattoo pigment particles.

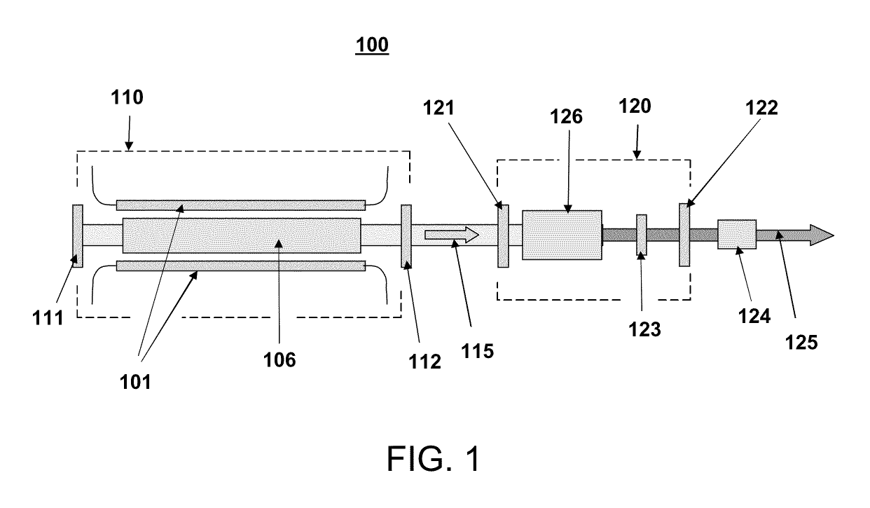

FIG. 1 is a schematic drawing of a laser system 100 including a solid state laser 110 pumping a second solid state laser 120. Laser gain materials 106 and 126 of the solid state lasers 110 and 120, respectively, can be made in the shape of a round rod, but other configurations can also be used, such as, for example, slabs and cubes. The solid state laser 110 can be a transition metal laser such as, for example, an Alexandrite laser. The solid state laser 110 can be pumped by one or more flashlamps 101. The cavity of the solid state laser 110 can include a high reflecting mirror 111 and an output coupler mirror 112. The output coupler mirror 112 can couple an output beam 115 from the cavity of the first solid state laser 110. The solid state laser 120 can be a rare-earth laser such as, for example, neodymium-YAG (Nd:YAG). The solid state laser 120 can be pumped by a portion of the output beam 115. The output beam 115 can include either long, gain switched pulses, or short, Q-switched, pulses. The temporal profile of the output beam 125 from the laser cavity of the solid state laser 120 has been experimentally found to closely match the temporal profile of the output beam 115 for both long-pulse and/or short-pulse pumping. In addition, a Q-switched solid state laser can be constructed so that long pulses can be produced by not energizing the Q-switching device.

The cavity of the solid state laser 120 can include a high reflecting mirror 121 and an output coupler mirror 122. The output coupler mirror 122 can couple an output beam 125 from the cavity of the solid state laser 120. In one embodiment, the cavity of the solid state laser 120 can include a q-switching element 123 such as, for example, a Cr⁴⁺:YAG (Cr:YAG) element. In some embodiments, a frequency doubling crystal 124 such as, for example, KTP, can be positioned in the path of the output beam 125.

In one embodiment, the solid state laser gain materials 106 and/or 126 can be directly coated on both ends with coatings of appropriate transmission and reflection properties to form the reflecting mirrors 111 and 121 and the output coupling mirrors 112 and 122. For a Alexandrite/Nd:YAG system, the reflecting mirror 121 and the output coupler mirror 122 can allow both double-pass pumping at about 755 nm and/or laser output at about 1064 nm. The absorption coefficient of Nd:YAG at about 755 nm can be about 2 cm⁻¹. Therefore, a solid state laser 120 with a Nd:YAG gain medium 1.15 cm long can absorb approximately 99% of the pump energy in a double-pass configuration.

Coupling the output laser beam 115 of the solid state laser 110 into the gain material 126 of the solid state laser 120 can be accomplished in a variety of ways such as, for example, end-pumping, as illustrated in FIG. 1. In one embodiment, the laser gain material 126 of the solid state laser 120 can be made larger than the laser beam 115 so that the solid state laser 120 can be pumped directly without any manipulation of the pump beam 115. The dimensions of the end of the solid state laser gain material 126 can be made slightly larger than the laser beam 115 so that the solid state laser 120 can be positioned directly in the path of the beam 115. Alternatively, the beam 115 can be shaped with mirrors, lenses, and/or other optical components to optimize the pump volume within the solid state laser 120. Either solid state laser 110 or solid state laser 120 can be mounted on a translation stage so that the system output beam 125 can be switched between about 755 nm and about 1064 nm simply by moving one of the solid state lasers. Translational stages can be used in systems where output beam 115 and/or output beam 125 are focused into an optical fiber for delivery of the treatment beam. Both output beams 115 and 125 can be more efficiently coupled into the fiber without any positional adjustment of the fiber coupling components (e.g., lens (not shown)).

Both Alexandrite and Nd:YAG lasers can be used in dermatological applications. Therefore, the selection of Alexandrite and Nd:YAG as the solid state lasers 110 and 120, respectively, in the configuration illustrated in FIG. 1 can allow for complementary applications in a single system. The q-switched versions of both Alexandrite and Nd:YAG lasers, for example, can be used to remove tattoos. Due to the different optical absorption of the various colors of tattoo pigment, an Alexandrite laser can remove blue and green tattoos, while a Nd:YAG laser can remove black tattoos. The second harmonic of a Nd:YAG laser, about 532 nm, can remove red tattoos. Likewise, long-pulse versions of both Alexandrite and Nd:YAG lasers can be effective for hair removal. However, because the wavelengths of Alexandrite and Nd:YAG lasers have different optical absorption in melanin, an Alexandrite laser, at about 755 nm, can be better for treating light-skin patients while a Nd:YAG laser, at about 1064 nm, can be better for dark-skin patients.

The absorption spectra of Nd:YAG has a continuous band of lines ranging from about 725 nm to about 770 nm. Therefore, the non-tuned output of a free-running Alexandrite laser, at about 755 nm, can be used to pump a Nd:YAG laser. An important absorption line in the Nd:YAG is about 2 or 3 nm wide centered at about 755 nm. A stronger but narrower peak is centered at about 750 nm. Furthermore, there are wavelengths within the 725 nm to 770 nm band where excited state absorption can occur. However, there is very little excited state absorption at 755 nm, making it an attractive pump wavelength. The efficiency of the conversion of 755 nm to 1064 nm can be affected mostly by the quantum defect, which is about 30%. There can be another small loss, e.g., less than 5% percent in Nd:YAG, due to scattering effects.

Laser pumping can be particularly attractive for lasers that are difficult to pump with other conventional sources such as, for example, laser diodes and flashlamps. As an example, it can be difficult to generate high peak powers from Nd:YAG at 946 nm, which is one of the laser lines of Nd:YAG. The 946 nm is a three-level laser transition which requires a high pump rate to reach threshold. Flashlamp pumping can be inadequate due to poor brightness of the source, while diode lasers can essentially be continuous wave sources and not suitable for high peak power applications.

When end-pumped by a Q-switched Alexandrite laser emitting a 50 ns pulse at 755 nm, for example, Nd:YAG can readily lase at 946 nm, emitting a similarly short pulse with hundreds of milli-Joules of energy, corresponding to several MW of peak power. In another example, when end-pumped with 25 Joule, 3 millisecond pulses from a free-running, gain-switched Alexandrite laser, an output of 6 Joules at 946 nm can be emitted by the Nd:YAG laser.

Although the pump beam can be absorbed by the gain medium, high absorption is not preferred in all embodiments. For example, some heat can be generated in a rare-earth gain medium by laser pumping, although the amount of heat can be much less than that deposited in the gain medium by flashlamp pumping. Nevertheless, the size of the gain medium can be chosen so that the heat can be removed fast enough to limit the temperature rise in the gain medium. The length of the gain medium and the magnitude of the absorption can be chosen so that the heat generation is distributed fairly evenly through the medium. In some cases, for example, the wavelength emitted by the pump laser can be tuned in order to adjust the absorption of the pump beam by the rare-earth laser.

Absorption spectra show that Nd:YVO₄, and Nd:GdVO₄ can also be excited by a free running Alexandrite laser. A tunable Alexandrite laser, from about 700 nm to about 818 nm, can be used to excite other laser gain materials such as, for example, Nd:YAP, Er:YAG, and/or Tm:YAG. Ti:sapphire, with a broader tunable range from about 700 nm to about 1050 nm, can also be used to pump Ho:YAG. The approximately 2.94 micron wavelength output of a Er:YAG laser has high optical absorption by water and can therefore be used to ablate a thin layer of the epidermis for removing some of the effects of aging and sun damage.

In some embodiments, Tm:YAG can provide laser output when pumped by a free-running Alexandrite laser. For example, when the thulium concentration is at 6%, a gain length of two inches absorbed 95% of the pump energy with a double-pass pump configuration. The thulium laser can produce 8 Joules of approximately 2 micron laser output when pumped with a 25 Joule, 755 nm laser beam. In this case, about 15 Joules is deposited in the laser rod. The long length of the laser rod can provide sufficient surface area from which to extract the heat between pulses.

Like the Er:YAG above, the approximate 2 micron output of a Tm:YAG laser can be usable for improving the appearance of aged skin. Tm:YAG has the advantage that the wavelength of the output is tunable from about 1.93 microns to about 2.10 microns. The wavelength can be adjusted so that the depth of penetration in the skin can be selected over a range of about 110 micron to about 600 microns.

The laser system 100 can treat a patient at either or both of two wavelengths produced by solid state lasers 110 and 120 at the same or two different pulse durations. For example, a Q-switched Alexandrite laser without a tuning element as solid state laser 110 can produce approximately 50 nanosecond pulses at about 755 nm. The output beam 115 in this configuration can be used to treat the patient or to pump a solid state laser 120 such as Nd:YAG, in which case approximately 50 nanosecond pulses at about 1064 nm can be produced and can be used to treat the patient. By not energizing or not including a Q-switching element in either cavity of solid state lasers 110 and 120, the laser system 100 can also be used to treat the patient with long-pulses of either wavelength. In this configuration, the duration of the long pulses can be determined by the duration and output power of the energy pulses produced by the one or more flashlamps 101.

The laser system 100 can realize one or more of the following advantages over a conventional Q-switched Nd:YAG laser. The duration of the pulse generated by a conventional Q-switched Nd:YAG laser is about 10 nanoseconds. At effective treatment energies, the peak power can be so high that it cannot be transmitted though an optical fiber without damaging the fiber. A conventional Q-switched Nd:YAG laser system, therefore, typically has expensive and inconvenient articulated-arm beam delivery systems to overcome this problem. The 50 nanosecond pulses generated, for example, by the laser system 100 can be transmitted by optical fiber, a simpler and less expensive design. Pulses generated by the laser system 100 can also be as long as 100 nanoseconds. Furthermore, a higher output energy is possible with laser system 100. Q-switched operation can require that energy be stored in the laser cavity. But amplified spontaneous emission (ASE) can limit the amount of energy that can be stored in a Nd:YAG cavity, resulting in limited output. Gain switched operation in laser system 100, however, does not have this problem because of the short duration of the pumped state of the laser gain material and of the high Q of the cavity.

In various embodiments, frequency doubling can be used to obtain about a 532 nm output beam 125. For example, high peak power of 50 nanosecond Nd:YAG pulses from solid state laser 120 can enable efficient second-harmonic conversion of the about 1064 nm wavelength to about 532 nm. Generation of the second-harmonic can be accomplished with the frequency doubling crystal 124, such as, for example, a KTP crystal. The output laser beam from solid state laser 120 laser can be polarized in order to maximize the efficiency of the wavelength conversion within the frequency doubling crystal 124. A polarizing element can be installed in the cavity of solid state laser 120. In an alternative embodiment, a different host material for solid state laser 120 can be selected. Both Nd:YVO₄, and Nd:GdVO₄ can produce linearly polarized outputs at about 1064 nm and about 1063 nm, respectively. The long-pulse 1064 nm beam may not be efficiently frequency doubled because the peak power is low. This problem can be overcome by repetitively Q-switching the solid state laser 120 or the solid-state laser 110. Either active or passive Q-switching can accomplish repetitive Q-switching. A Cr⁴⁺:YAG passive Q-switch can also be placed in the resonator cavity of solid state laser 120 to generate a train of high peak power pulses that can be efficiently frequency doubled to about 532 nm.

In some embodiments, the pump beam 115 can be coupled into the solid state laser 120 using a fiber optic coupling system. First, one or more lenses or other optical components can converge at least a portion of the pump beam 115 into an optical fiber (not shown) though which a portion of the pump beam 115 can be transmitted. The beam exiting the distal end of the optical fiber can be a divergent beam, which can be directed into the gain material of solid state laser 120 or it can be shaped and/or collimated by one or more lenses or other optical components before being directed into the gain material of solid state laser 120.

In one embodiment, a diode laser output at 808 nm can be used for hair removal and for pumping a Nd:YAG laser. The diode laser system can include an optical system to optimize the divergence of the diode laser beam for treating hair and pumping the Nd:YAG laser.

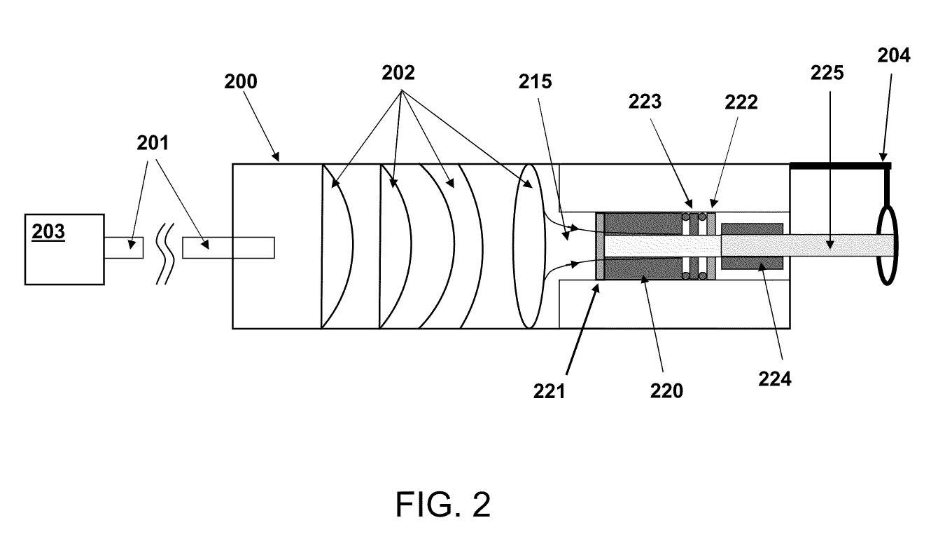

FIG. 2 is a schematic drawing of a handpiece 200 including a solid state laser 220 pumped via an optical fiber 201 by another solid state laser 203. A spacer 204 can space the handpiece 200 from a skin surface. In various embodiments, the handpiece 200 can include one or more optical components 202 for coupling at least a portion of the output beam 215 from the optical fiber 201 into the cavity of the solid state laser 220. The cavity of the solid state laser 220 can include a high reflecting mirror 221 and an output coupler mirror 222. In one embodiment, the cavity of the solid state laser 220 can include a Q-switching element 223. In certain embodiments, the handpiece 200 can include a frequency doubling crystal 224. The output beam 225 of the handpiece can be optically modified by an optical component 202 before it is used to treat the skin of a patient. In yet a further embodiment, the solid state laser 220 in the handpiece 200 can include an Er:YAG laser. In some embodiments, the solid state laser 220 in the handpiece 200 can include a Tm:YAG laser with or without a wavelength tuning device such as, for example, a birefringent tuner.

An alexandrite-pumped-neodymium laser system can be useful for a variety of medical applications, and in particular, dermatology. The three treatment wavelengths and two pulse durations capable of being produced by the laser system 100 can provide a range of six spectrally and temporally selective treatment modes thereby making this system clinically effective for a large range of medical conditions. The efficient conversion of electrical input energy to laser output energy at all three wavelengths can allow the design of competitively sized and priced laser products. Products based on sub-sets of the elements described herein can also be clinically useful and commercially viable.

To minimize thermal injury to tissue surrounding an eye and/or to an exposed surface of the target region, the delivery system (e.g., handpiece 200) can include a cooling system for cooling before, during and/or after delivery of radiation. Cooling can include contact conduction cooling, evaporative spray cooling, convective air flow cooling, or a combination of the aforementioned. In one embodiment, the handpiece 200 includes a skin contacting portion that can be brought into contact with the skin. The skin contacting portion can include a sapphire or glass window and a fluid passage containing a cooling fluid. The cooling fluid can be a fluorocarbon type cooling fluid, which can be transparent to the radiation used. The cooling fluid can circulate through the fluid passage and past the window to cool the skin.

A spray cooling device can use cryogen, water, or air as a coolant. In one embodiment, a dynamic cooling device can be used to cool the skin (e.g., a DCD available from Candela Corporation). For example, the delivery system can include tubing for delivering a cooling fluid to the handpiece 200. The tubing can be connected to a container of a low boiling point fluid, and the handpiece 200 can include a valve for delivering a spurt of the fluid to the skin. Heat can be extracted from the skin by virtue of evaporative cooling of the low boiling point fluid. The fluid can be a non-toxic substance with high vapor pressure at normal body temperature, such as a Freon or tetrafluoroethane.

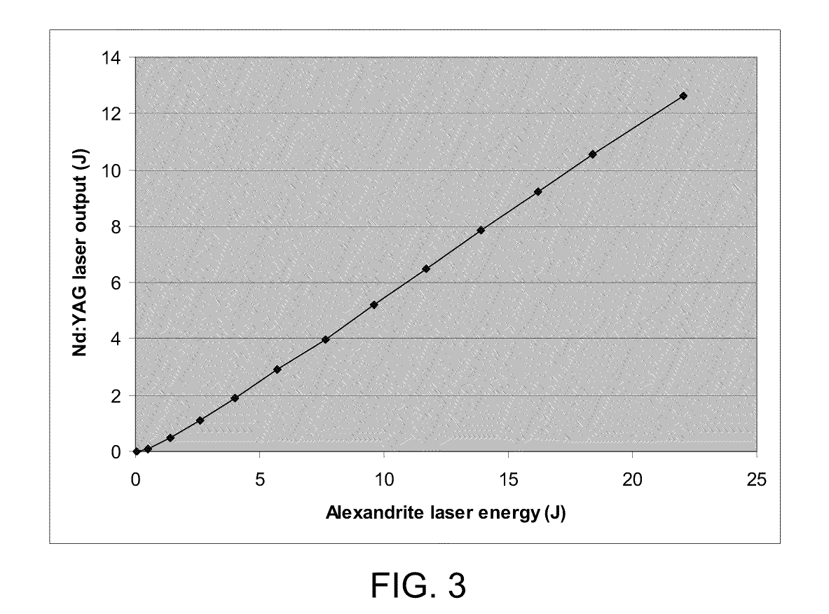

FIG. 3 shows laser output of a Nd:YAG laser pumped by an Alexandrite laser.

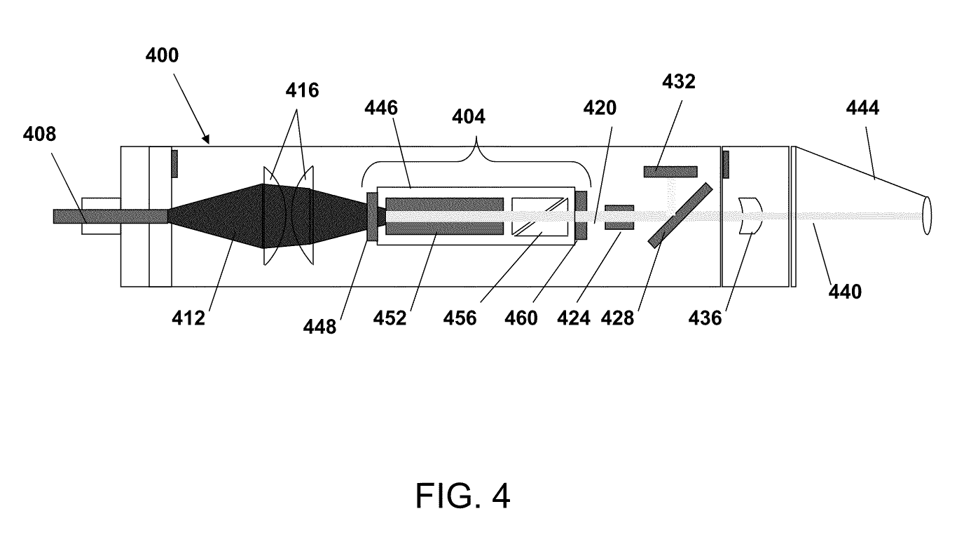

FIG. 4 shows a laser handpiece 400 including a precision tube assembly 404. An optical fiber 408 delivers radiation 412 through focusing lens 416 to the precision tube assembly 404. Radiation 420 from the precision tube assembly 404 is directed to a harmonic generator 424 (e.g., a KTP crystal). A beam splitter 428 separates pump radiation from the harmonic. The pump radiation is directed to a beam dump 432, while the harmonic is directed to a lens 436, which directs radiation 440 to a skin surface. A spacer 444 can space the handpiece from the skin surface. Lens 436 can converge, collimate, or diverge optical radiation.

The precision tube assembly 404 includes a tubular member 446, a high reflector 448, a laser crystal 452, a polarizer 456, and an output coupler 460. The high reflector 448 can be coated on the laser crystal 452, or can be a separate optic spaced from the laser crystal 452. As shown in FIG. 1, a q-switching element, e.g., a Cr⁴⁺:YAG crystal, can be used in the laser resonator to provide higher peak power. In certain embodiments, a q-switching element takes the place of the polarizer 456.

The tubular member can have parallel faces, which can register the faces of the high reflector 448 and the output coupler 460 parallel or substantially parallel. In certain embodiments, the tubular member can have faces that are offset from parallel at a predetermined angle. The high reflector 448, the output coupler 460, and/or one or more faces of the laser crystal 452 can be wedged to compensate for the predetermined angle of the offset.

Tubular member 446 need not be a hollow cylinder. Sectional geometries including circular, triangular, square, pentagonal, or any suitable polygonal geometry. A geometry that has an angled inner surface can be used to align the laser crystal 452 within the tubular member 446. The laser crystal 452 can be anti-reflection coated on one or both faces.

In certain embodiments, the polarizer 456 can be a Brewster plate or a prism polarizer. In some embodiments, a polarizer 456 need not be used and the laser crystal 452 can be formed from a self-polarizing material.

In one embodiment, a 532 nm handpiece includes a frequency doubled Nd:YAG laser pumped by a q-switched alexandrite laser. An optical fiber carries the 755 nm light from the base laser to the handpiece at the distal end where the wavelength conversion from 755 nm to 532 nm occurs. The 755 nm light is focused by a pair of focusing lenses to form an image of the fiber facet onto an Nd:YAG rod situated in the handpiece. The Nd:YAG rod is sandwiched between appropriately coated mirrors (e.g., the high reflector and output coupler) to form a laser resonator. When pumped at 755 nm, the laser rod can be made to lase at 1064 nm with high efficiency approaching 70%. A Brewster plate polarizes the 1064 nm output beam is polarized. The 1064 nm beam is then incident on a KTP crystal that is aligned for optimum phase matching for second harmonic generation. The KTP crystal converts 1064 nm to 532 nm efficiently. A dichroic beam-splitter separates the 532 nm beam from the residual unconverted 1064 nm. The 532 nm beam is appropriately shaped and sized using lenses and is incident on the skin during treatment. The unconverted 1064 nm that is rejected by the beam-splitter is safely absorbed in a beam dump within the handpiece. The spot size on the Nd:YAG rod can be about 4.5 mm. In certain embodiments, the cavity length can be 30-40 mm.

A precision tube assembly achieves the required parallelism of the mirrors by registering the mirrors against the tubular member having parallel or substantially parallel faces. The parallelism can be about five arc seconds, translating to an error of only 0.25 microns in length of the tube across a 10 mm diameter. The high reflector is a separate optic spaced about 1 mm away from the Nd:YAG rod.

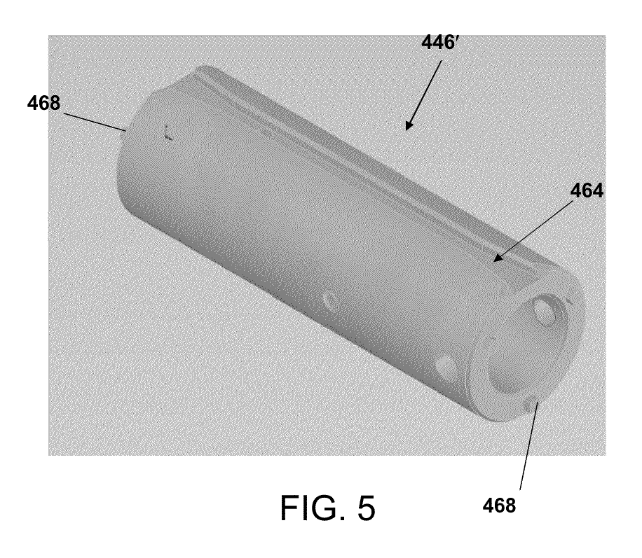

FIG. 5 shows an embodiment of a tubular member 446′ including a channel 464 for engagement and/or alignment with a laser handpiece. Engagement between the channel 464 and a pin or alignment feature of the laser handpiece prevents rotation of the tubular member 446′ in the laser handpiece. The tubular member 446′ includes one or more raised regions 468 on one or both ends. The surface area of each raised region 468 can be about 1 mm×1 mm, and the raised region 468 can extend from the surface of the tubular member 446′ by about 0.25 mm to about 5 mm. In certain embodiments, the raised region 468 extends from the surface of the tubular member 446′ by about 1 mm. In some embodiments, the tubular member 446′ includes three raised regions 468 equally or substantially equally spaced around the circumference of the tubular member 446′, although the raised regions can be irregularly spaced.

The raised regions 468 can define the plane to which a high reflector and an output coupler are registered. The two planes defined by the raised regions 468 on each face can be lapped parallel to better than 5 arc seconds. Thus, when the high reflector and output coupler are pressed against the tubular member 446′, they are parallel or substantially parallel to approximately the same degree. In certain embodiments, the planes of the raised regions 468 can be offset from parallel at a predetermined angle. The high reflector 448, the output coupler 460, and/or one or more faces of the laser crystal 452 can be wedged to compensate for the predetermined angle of the offset. Raised regions 468 allow for minimum contact between the tubular member 446 and the high reflector 448 and/or the output coupler 460, which can aid alignment of the precision tube assembly 404.

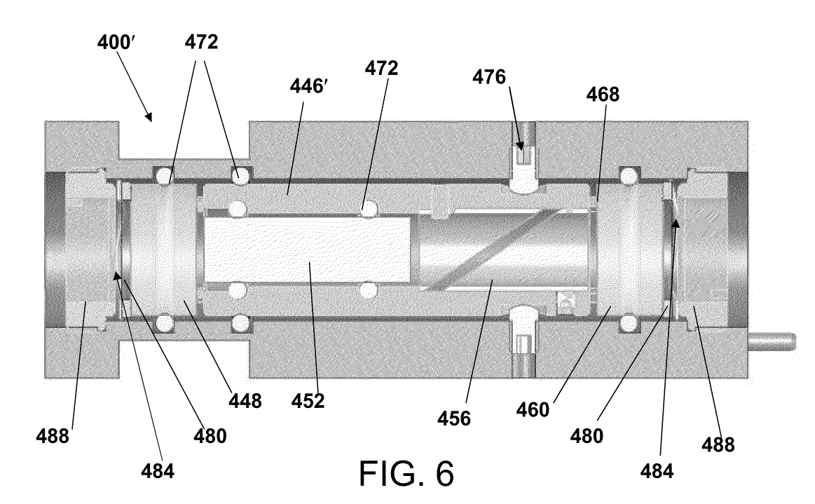

FIG. 6 shows a sectional view of the tubular member 446′ disposed in a laser handpiece 400′. The tubular member 446′ engages the laser crystal 452 with O-rings 472, and the laser handpiece 400′ engages the tubular member 446′ with o-rings 472. Set screws 476 can hold the tubular member 446′ in place. Adjustment of the set screws 476 can tilt the tubular member 446′ to steer the radiation.

The high reflector and the output coupler can float on O-rings 472 to facilitate alignment of the mirrors. A member 484 can be used to place a load on the mirrors to move them into contact with and/or press them against the tubular member 446′. Member 484 can press a spacer 480, which can distribute the load of the spacer 484. Member 484 can be a washer (e.g., a wavy washer having a non-planar shape). The spacer 480 can be a flat spacer with a hollow center to permit laser radiation to be transmitted through its central portion. The wavy washer can be positioned behind each mirror to provide a positive force (e.g., a spring load) to ensure constant positive pressure on the mirrors against the raised regions of the tubular member 446′. Locking rings 488 can pressing on the member 484 (e.g., wavy washers). Locking rings 488 can bottom out on a step to ensure that the pressure on the member 484 is independent of the torque used to tighten the locking ring 488.

The invention has been described in terms of particular embodiments. The alternatives described herein are examples for illustration only and not to limit the alternatives in any way. The steps of the invention can be performed in a different order and still achieve desirable results. Other embodiments are within the scope of the following claims.

What is claimed:

1. A laser handpiece comprising:
a tubular member having a first end surface and a second end surface, at least one raised region being formed on each end surface of the tubular member;
a first optical component engageable with the at least one raised region associated with the first end surface of the tubular member; and
a second optical component engageable with the at least one raised region associated with the second end surface of the tubular member, the tubular member adapted to align the first optical component and the second optical component parallel or substantially parallel.

2. The laser handpiece of claim 1 wherein the at least one raised region is adapted to align the first optical component and the second optical component to within about five arc seconds.

3. The laser handpiece of claim 1 wherein the at least one raised region comprises a plurality of raised regions spaced substantially evenly around each end surface of the tubular member.

4. The laser handpiece of claim 1 wherein the tubular member comprises a channel engageable with an alignment feature of the laser handpiece to register faces of the first optical component and the second optical component parallel or substantially parallel.

5. The laser handpiece of claim 1 wherein the at least one raised region on the first end surface defines a first plane and the at least one raised region on the second end surface defines a second plane, and the first plane and the second plane are parallel or substantially parallel.

6. The laser handpiece of claim 5 wherein the first plane and the second plane are lapped parallel to better than five arc seconds.

7. The laser handpiece of claim 1 further comprising a washer pressing against a spacer to provide a force against an optical component to contact between the optical component and the at least one raised region of the tubular member.

8. The laser handpiece of claim 7 further comprising a locking ring engaging the laser handpiece to apply force to the washer.

9. The laser handpiece of claim 1 wherein the first optical component is a high reflector and the second optical component is an output coupler.

10. The laser handpiece of claim 1 further comprising a Brewster plate disposed within the tubular member.

11. A laser system comprising:
a first solid-state laser for producing a first output beam; and
a laser handpiece in optical communication with the first solid-state laser, the laser handpiece comprising:
a tubular member having a first end surface and a second end surface, at least one raised region being formed on each end surface of the tubular member;
a first optical component engageable with the at least one raised region associated with the first end surface of the tubular member;
a second optical component engageable with the at least one raised region associated with the second end surface of the tubular member, the tubular member adapted to align the first optical component and the second optical component parallel or substantially parallel;
a second laser gain medium within the tubular member for producing a second output beam, the second solid state adapted to receive a first part of the first output beam and generate excitation in the second laser gain medium to produce the second output beam.

12. The laser handpiece of claim 11 further comprising a nonlinear frequency converter for generating, based on laser radiation received from the second laser gain medium, the second output beam.

13. The laser handpiece of claim 11 wherein the first solid-state laser comprises a first host material, the first host material comprising sapphire, beryl, chrysoberyl, LiSAF, forsterite, or any combination thereof.

14. The laser handpiece of claim 13 wherein the first host material is doped with a transition metal comprising Cr or Ti.

15. The laser handpiece of claim 11 wherein the second laser gain medium comprises a rare-earth doped gain medium comprising YAG, YAP, YVO₄, YLF, YSGG, GSGG, FAP, GdVO₄, KGd(WO₄)₂, SFAP, glass, ceramic, or any combination thereof.

16. The laser handpiece of claim 15 wherein the second laser gain medium is doped with rare-earth ions comprising Nd, Yb, Er, Ho, Th, Sm, Ce, or any combination thereof.

17. The laser handpiece of claim 11 wherein:
the first solid-state laser comprises a Q-switched alexandrite laser for producing a first output beam, the wavelength of the first output beam being about 755 nm; and
the second laser gain medium comprises Nd:YAG for producing the second output beam of about 1064 nm.

18. The laser handpiece of claim 17 wherein a KTP crystal is adapted to receive the second output beam and generate a third output beam of about 532 nm.

19. An alignment tube for a laser handpiece, comprising:
a tubular member; and
at least one raised region formed on an end circumference of the tubular member, the at least one raised region adapted to align an optical component in the laser handpiece to better than about five arc seconds.
